(19) United States
(12) Patent Application Publication
(10) Pub. No.: US 2007/0042394 A1
(43) Pub. Date: Feb. 22, 2007

(54) Apparatus for producing a DNA doped carbon cluster and method for producing the same

(76) Inventors: Rikizo Hatakeyama, Sendai-shi (JP); Toshiro Kaneko, Sendai-shi (JP); Takeru Okada, Sendai-shi (JP); Kazuhiko Hirai, Sendai-shi (JP); Yasuhiko Kasama, Sendai-shi (JP); Kenji Omote, Sendai-shi (JP)

Correspondence Address: YOUNG & THOMPSON, 745 SOUTH 23RD STREET, 2ND FLOOR, ARLINGTON, VA, 22202, US

(21) Appl. No.: 11/338,636

(22) Filed: Jan. 25, 2006

(30) Foreign Application Priority Data
May 13, 2005 (JP) 2005-140660
Dec. 20, 2005 (JP) 2005-366297

(51) Int. Cl. C12Q 1/68; C07H 21/04; B01J 19/08

(52) U.S. Cl. 435/6; 536/24.3; 977/924; 422/186.05

(57) ABSTRACT

The object of the present invention is to provide an apparatus for producing a DNA doped carbon cluster capable of introducing DNA into the cavity of a carbon cluster. An apparatus for producing a DNA doped carbon cluster comprising a radio frequency current applying electrode which is composed of a porous material or a wire mesh capable of retaining a solution containing DNA, a grounding electrode placed opposite to the radio frequency current applying electrode, and a power source for supplying a radio frequency output to the radio frequency current applying electrode, wherein the grounding electrode bears hollow carbon clusters on its surface.

### BACKGROUND OF THE INVENTION

1. Field of the Invention

The present invention relates to an apparatus for producing a DNA doped carbon cluster obtained by introducing DNA into a carbon cluster such as fullerene or carbon nanotube, and method for producing such a DNA doped carbon cluster, and to a DNA doped carbon cluster produced with the apparatus according to the method.

2. Description of the Related Art

Recently, carbon clusters such as fullerenes, carbon nanotubes, carbon nanohorns, bucky onions, etc., attracts attention, and the apparatuses and method responsible for their production also attract attention (see, for example, Japanese Unexamined Patent Application Publication No. 2003-300713).

In the mean time, researches on the methods for introducing a metal ion into the central void of a carbon cluster and for evaluating the utility of a product incorporating such a carbon cluster have been pursuit vigorously (see, for example, Japanese Unexamined Patent Application Publication Nos. 10-324513 and 2004-099417, and T. Shimada et al., “Transport properties of C78, C90 and Dy@C82 fullerenes-nanopeapods by field effect transistors,” Physica E, Vol. 21(2004), pp. 1089-1092).

On the other hand, fifty years have passed since the discovery of the stereoscopic structure of DNA, and practical application of the knowledge accumulated during the period into new technical fields advanced so rapidly that the knowledge is utilized not only for recombinant DNA techniques but also for DNA typing and construction of functional electronic apparatuses.

For example, an attempt has been made to adsorb DNA onto an aluminum electrode by electrophoresis (see, for example, M. Ueda et al., “Atomic force microscopy observation of deoxyribonucleic acid stretched and anchored onto aluminum electrodes,” Jpn. J. Appl. Phys., Vol. 38(1999), pp. 2118-2119).

The present inventors also tried to introduce DNA into a carbon cluster in a liquid using an electrophoresis method (see Japanese Patent Application No. 2004-278816).

### SUMMARY OF THE INVENTION

DNA has a double helical structure composed of five kinds of elements (C, H, N, O, P), and readily loses its normal function as a result of the destruction or degradation of its structure when it is heated or exposed to the working of various chemicals. As seen from this, DNA is very susceptible to changes in the external environment, and thus handling of DNA requires considerable attention.

In contrast, a carbon cluster, particularly a carbon nanotube has a high mechanical strength, even though it has a hollow structure, and is more resistant to chemicals than DNA. Thus, the carbon cluster has a structure suitable for working as a protective receptacle for DNA. Moreover, it is possible to prepare a carbon cluster having a size slightly larger than or as large as the size of DNA.

If it is possible to prepare a carbon nanotube having a size as large as that of DNA and to store DNA in its central void, the protective function of the carbon nanotube will effectively compensate for the weak-point of DNA, that is, its susceptibility to the effects from external environment, and DNA protected in such a nanotube will have new and promising applications.

The present invention aims to provide an apparatus for producing a DNA doped carbon cluster containing DNA in its central void, and method for producing such a DNA doped carbon cluster, and to a DNA doped carbon cluster produced with the apparatus according to the method.

To achieve the above object, an aspect of the invention as described in Claim 1 relates to an apparatus for producing a DNA doped carbon cluster comprising an electrode for applying radio frequency (RF) current which is composed of a porous material or a wire mesh capable of retaining a solution containing DNA, a grounding electrode placed opposite to the RF current applying electrode, and a power source for supplying an RF output to the RF current applying electrode, wherein the grounding electrode bears hollow (open-end) carbon clusters on its surface.

Another aspect of the invention as described in Claim 2 relates to an apparatus as described in Claim 1 for producing a DNA doped carbon cluster wherein the RF current applying electrode composed of a wire mesh comprises a laminated electrode composed of two kinds of wire meshes having different mesh densities.

A yet another aspect of the invention as described in Claim 3 relates to an apparatus as described in Claim 1 or 2 for producing a DNA doped carbon cluster wherein the solvent of the DNA containing solution is any one chosen from the group consisting of pure water, distilled water and liquid paraffin.

A further aspect of the invention as described in Claim 4 relates to an apparatus for producing a DNA doped carbon cluster comprising an RF current applying electrode composed of a porous material or a wire mesh capable of retaining a DNA containing solution, a grounding electrode placed opposite to the RF current applying electrode, and a power source for supplying an RF output to the RF current applying electrode, wherein the solvent of the DNA containing solution is liquid paraffin.

A still further aspect of the invention as described in Claim 5 relates to an apparatus as described in any one of Claims 1 to 4 for producing a DNA doped carbon cluster wherein the grounding electrode and the RF current applying electrode are arranged on a silicon substrate.

A still further aspect of the invention as described in Claim 6 relates to an apparatus as described in any one of Claims 1 to 4 for producing a DNA doped carbon cluster wherein the grounding electrode and the RF current applying electrode are arranged on a glass substrate.

A still further aspect of the invention as described in Claim 7 relates to a method for producing a DNA doped carbon cluster comprising the step of applying an RF output to a space between the RF applying electrode retaining DNA and the grounding electrode placed opposite to the RF applying electrode which bears hollow carbon clusters on its surface, so as to generate plasma in that space, thereby producing DNA doped carbon clusters.

A still further aspect of the invention as described in Claim 8 relates to a method for producing a DNA doped carbon cluster comprising the step of applying an RF power to a space between the RF applying electrode retaining DNA-containing liquid paraffin and the grounding electrode placed opposite to the RF applying electrode so as to generate plasma in that space, thereby producing DNA doped carbon clusters.

A still further aspect of the invention as described in Claim 9 relates to a method as described in Claim 7 or 8 for producing a DNA doped carbon cluster wherein generation of plasma is achieved under the atmospheric pressure.

A still further aspect of the invention as described in Claim 10 relates to a DNA doped carbon cluster produced with an apparatus as described in any one of Claims 1 to 6.

A still further aspect of the invention as described in Claim 11 relates to a DNA doped carbon cluster produced by a method as described in any one of Claims 7 to 8.

It is possible to introduce DNA fragments varied in size into a carbon cluster by varying as appropriate the size and shape of the receptive carbon cluster, or by choosing an appropriate kind of carbon cluster (fullerene, carbon nanotube, carbon nanohorn, bucky-onion, etc.). For example, a carbon nanotube can contain DNA fragments having a length up to 20 to 40 nm in its void. If the length of a DNA fragment to be shut in is determined, it will be possible to choose a carbon cluster to serve as a suitable receptacle for the DNA fragment, and to produce a DNA doped carbon cluster from them by using the inventive method.

According to the present invention, pure water is defined to be one close to the purified water which is obtained by treating water chemically and physically to become clear, and depriving the resulting water of solutes (containing colloids). Distilled water is defined here to be water obtained by boiling water to convert it into a steam, and cooling the steam to condense it into water. The above definitions are the same with those commonly used.

According to the aspects of the invention as described in Claims 1, 2 and 7, it is possible to introduce DNA into a carbon cluster with the aid of discharge, to acquire DNA having a high resistance to environmental disturbances under the protection of the retentive carbon cluster, and to apply the safely guarded DNA to the medicinal and therapeutic fields hitherto inaccessible owing to the vulnerability of naked DNA to the disturbing effects of external environment.

According to the aspect of the invention as described in Claim 3, reduction in the production cost of DNA doped carbon clusters will be expected, because the solvent used for dissolving DNA comprises pure water, distilled water, liquid paraffin, etc., which are available comparatively easily.

According to the aspects of the invention as described in Claims 4 and 8, further reduction in the production cost of DNA doped carbon cluster will be expected, because introduction of DNA into carbon clusters is achieved by using liquid paraffin while the formation of carbon cluster is in progress, thus obviating the need for the deliberate preparation of carbon clusters with both ends open.

According to the aspects of the invention as described in Claims 5 and 6, still further reduction in the production cost of DNA doped carbon clusters will be expected, because the manufacture of the electrode for applying a RF power is simplified.

According to the aspect of the invention as described in Claim 9, still further reduction in the production cost of DNA doped carbon clusters will be expected, because it is possible to produce DNA doped carbon clusters under the atmospheric pressure without requiring the use of a vacuum chamber.

According to the aspects of the invention as described in Claims 10 and 11, the range of fields in medicine and therapeutics in which DNA can be utilized will be widened, because it will be possible to mass produce DNA resistant to environmental disturbances economically.

### BRIEF DESCRIPTION OF THE DRAWINGS

FIG. 1 is a diagram for showing the outline of an apparatus for producing a DNA doped carbon cluster described in Example 1 of the present invention.

FIG. 2 is a diagram for illustrating the principle enabling the introduction of DNA into a carbon nanotube.

FIG. 3 is a diagram for showing the outline of an apparatus for producing a DNA doped carbon cluster described in Example 2 of the present invention.

FIG. 4 shows the result of plasma experiment performed using the apparatus for producing a DNA doped carbon cluster depicted in FIG. 3.

FIG. 5 is a diagram for showing the principle enabling the introduction of DNA into a carbon cluster.

FIG. 6 represents the results of spectroscopic analysis performed on plasma.

### DESCRIPTION OF THE PREFERRED EMBODIMENTS

The apparatus for producing a DNA doped carbon cluster and the method for producing the same according to the present invention are to be explained with reference to the drawings. The scope of the present invention shall not be limited by the following examples unless it departs from the spirit of the invention.

### EXAMPLE 1

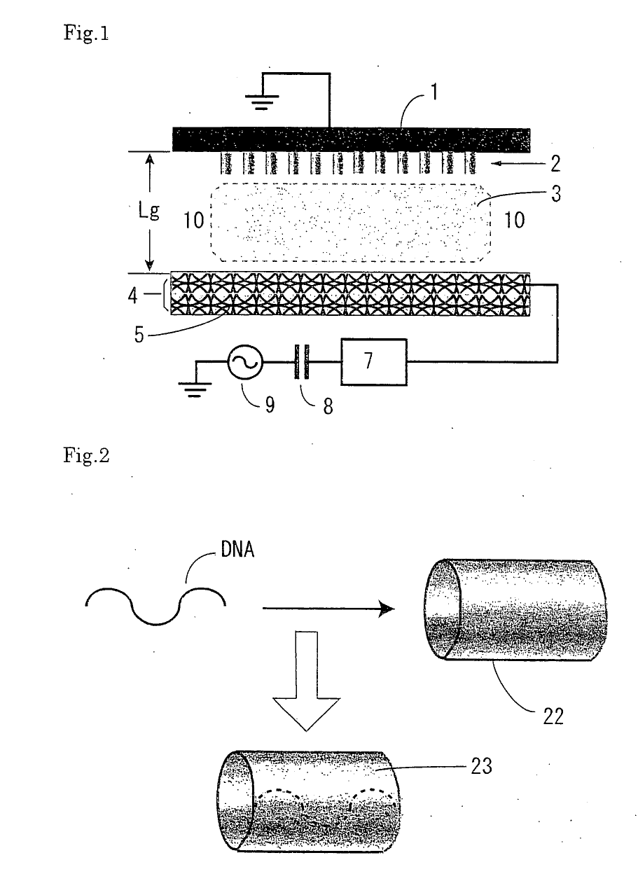

FIG. 1 is a diagram for showing the outline of an apparatus for producing a DNA doped carbon cluster representing a first embodiment of the present invention.

In FIG. 1, 1 represents a grounding electrode, 4 a wire mesh electrode for applying RF current, 5 DNA solution retained in the void of wire mesh electrode for applying RF current, 2 a carbon nanotube with both ends open, 3 helium plasma under the atmospheric pressure, 9 an RF power source working at 13.56 MHz, 8 a capacitor for intercepting direct current, and 7 a matching circuit. Members for reinforcing and fixing the grounding electrode 1 and RF current applying electrode 4 are omitted from the figure for simplicity.

DNA containing solution 5 is prepared in advance by dissolving DNA in liquid paraffin or in pure water or distilled water preferably at a concentration of 50 μg/ml. Liquid paraffin, or pure water or distilled water is chosen as the solvent of DNA because acquisition of them is comparatively easy and contamination by other elements than DNA can be easily prevented.

In this example, is used as a DNA sample a single strand DNA with a total length of about 5 nm consisting of a chain of 15 adenine bases which is commercially available. However, DNA fragments to be introduced into a carbon cluster are not limited in size to those whose total length is 5 nm or less. It is possible to introduce DNA fragments varied in size and shape into a carbon cluster by choosing an appropriate kind of carbon cluster (fullerene, carbon nanotube, carbon nanohorn, bucky-onion, etc.), or by varying the size and shape of carbon cluster.

Carbon nanotubes 2 with both ends open are adsorbed onto a surface of a copper grounding electrode 1 by a method as described later. A copper plate is chosen as an electrode material to which carbon nanotubes 2 are adsorbed, because it exhibits excellent high frequency characteristics, and highly pure copper material can be acquired easily at a comparatively low cost.

In this experiment, a copper plate is used as the grounding electrode 1. However, a silicon substrate having copper film on its surface may be used instead. Or, copper film may be formed on a glass substrate by a method such as electroplating or CVD.

The RF current applying electrode 4 is obtained by overlapping, one over the other, two copper or stainless steel meshes which are both commercially available. The two meshes have different mesh densities: one responsible for retaining DNA containing solution 5 has a mesh density of 10 meshes/inch, and the other facing the grounding electrode and responsible for electric discharge has a mesh density of 20 meshes/inch. The mesh having a lower mesh density (coarse mesh) is chosen for retaining the DNA-containing solution 5, because the mesh more effectively enhances the dispersion of the solution to lower the amount of solution per unit area, thereby facilitating the development of plasma, while the mesh having a higher mesh density (fine mesh) is placed opposite to the grounding electrode, because then droplets of DNA containing solution 5 ejected towards plasma become smaller in size as desired. Two wire meshes are fixed on a glass plate. However, they may be fixed on a silicon substrate instead.

In this example, one mesh having a mesh density of 10 meshes/inch is placed in advance on a glass plate, a DNA containing solution 5 is transferred to a space overlying the mesh using a needle and syringe. Then the other mesh having a mesh density of 20 meshes/inch is laid over the assembly to be fixed there. However, the two meshes may be combined at first to be placed on a glass plate, and a necessary amount of DNA containing solution 5 may be supplied over the assembly using a needle and syringe.

Carbon nanotubes prepared by the present inventors in a pilot experiment were treated by a method as described by T. Shimada et al. (supra) (carbon nanotubes were put in a glass ampoule, and heated for 2 days in an atmosphere kept at 10⁻² Pa and 450-500° C.) so that their both ends became open (hollow nanotubes). It is also possible to obtain hollow nanotubes by using nanotubes commercially available and treating them by a similar heating technique.

Hollow nanotubes in the form of a powder were dissolved in ethanol to be dispersed there to which ultrasounds were applied. A droplet of this suspension was applied to the surface of copper grounding electrode 1 to be dried there. Thus, the grounding electrode 1 was prepared which had hollow carbon nanotubes 2 attached on its surface.

As shown in FIG. 1, when helium gas 10 is flowed to accumulate in a space between the grounding electrode 1 and the RF current applying electrode 4, and the RF power source 9 was activated to apply 13.56 MHz RF output of about 10 W to the helium gas, helium plasma 3 was generated there under the atmospheric temperature. It has been known that DNA fragments are negatively charged in the plasma. Helium was used in this experiment because helium is ready to generate plasma under the atmospheric pressure. The output from the RF power source 9 may have a frequency of 2.45 GHz or a frequency in the range of microwave.

As seen from FIG. 2 illustrating the principle underlying the operation of the apparatus, DNA fragments vaporize from the RF current applying electrode 4, to be driven out via electric field developed on the surface of electrode 4, and drawn as negatively charged ions towards the plasma where they disperse to be attracted by the grounding electrode 1. The DNA fragments migrate towards the grounding electrode 1 to reach hollow nanotubes 22 there and enter into the cavity of hollow nanotubes. Thus, DNA doped carbon nanotubes 23 are obtained.

However, if the hollow nanotubes 23 doped with DNA have their both ends kept open, it would be difficult for them to completely satisfy the protective function assigned to them to serve as a protective receptacle for DNA. To meet this situation, it is only necessary to close, as needed, the open ends of those nanotubes 23 with fullerenes. For example, the grounding electrode 1 of FIG. 1 having DNA doped carbon nanotubes formed thereon may be immersed in a fullerene containing solution, another electrode to which negative voltage will be applied is placed opposite thereto, and a voltage is applied between the two electrodes. Then, fullerenes in the solution will migrate as a result of electrophoresis, to reach DNA doped hollow carbon nanotubes 23 there and close the open ends of those hollow nanotubes.

The method for producing DNA doped carbon nanotubes using a DNA containing solution obtained by dissolving DNA in a solvent and an apparatus operating based on the method have been described above. However, naked DNA, without being dissolved in any solvent, may be directly applied to the RF current applying electrode 4.

### EXAMPLE 2

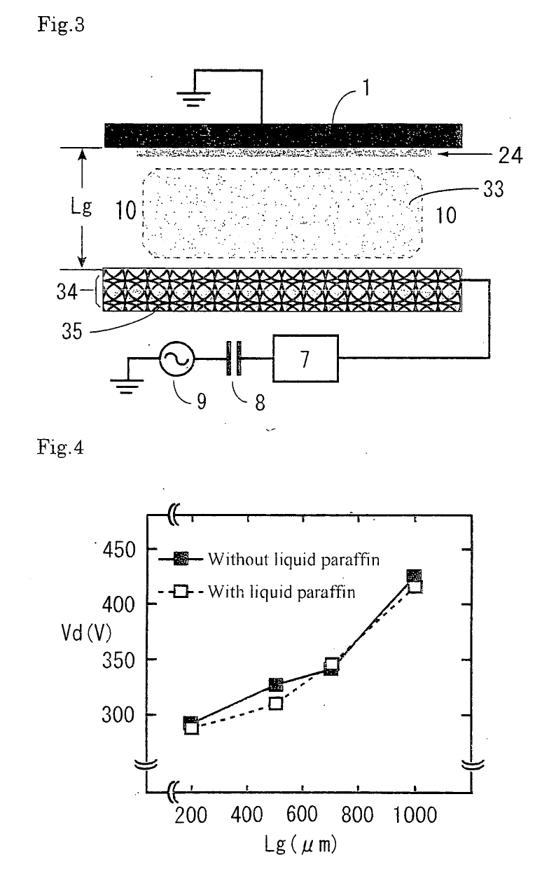

FIG. 3 is a diagram for showing the outline of an apparatus for producing a DNA doped carbon cluster representing a second embodiment of the present invention.

In FIG. 3, 1 represents a grounding electrode, 34 an electrode for applying an RF current, 35 a DNA containing solution kept at the bottom of the RF current applying electrode, 33 helium plasma present under the atmospheric pressure, 9 an RF power source working at 13.56 MHz, 8 a capacitor for intercepting direct current, and 7 a matching circuit. Members for reinforcing and fixing the grounding electrode 1 and RF current applying electrode 34 are omitted from the figure for simplicity.

In this example, one mesh having a mesh density of 10 meshes/inch is placed in advance on a glass plate, a DNA containing solution 35 is transferred to a space overlying the mesh using a needle and syringe. Then the other mesh having a mesh density of 20 meshes/inch is laid over the assembly to be fixed there. However, the two meshes may be combined at first to be placed on a glass plate, and a necessary amount of DNA containing solution 35 may be supplied over the assembly using a needle and syringe.

DNA containing solution 35 is prepared in advance by dissolving DNA in liquid paraffin (Product No. 164-00476, Wako Pure Chemicals) preferably at a concentration of 50 μg/ml. Liquid paraffin is chosen as the solvent of DNA because its acquisition is comparatively easy and contamination by other elements than DNA can be easily prevented. As a DNA sample is used a single strand DNA with a total length of about 5 nm consisting of a chain of 15 adenine bases which is commercially available.

DNA doped carbon clusters 24 are adsorbed onto a surface of a copper grounding electrode 1 as will be described later. A copper plate is chosen as an electrode material to which DNA doped carbon clusters 24 are adsorbed, because it exhibits excellent high frequency characteristics, and highly pure copper material can be acquired easily at a comparatively low cost.

In this experiment, a copper plate is used as the grounding electrode 1. However, a silicon substrate having copper film on its surface may be used instead. Or, copper film may be formed on a glass substrate by a method such as electroplating or CVD.

The RF current applying electrode 34 is obtained by overlapping, one over the other, two copper or stainless steel meshes which are both commercially available, or two nickel or iron meshes which serve as a catalyst during the formation of carbon clusters. The two meshes have different mesh densities: one responsible for retaining DNA containing solution 35 has a mesh density of 10 meshes/inch, and the other facing the grounding electrode and responsible for electric discharge has a mesh density of 20 meshes/inch. The mesh having a lower mesh density (coarse mesh) is chosen for retaining the DNA-containing solution 35, because the mesh more effectively enhances the dispersion of the solution to lower the amount of solution per unit area, thereby facilitating the development of plasma, while the mesh having a higher mesh density (fine mesh) is placed opposite to the grounding electrode, because then droplets of DNA containing solution 35 ejected towards plasma become smaller in size as desired. Two wire meshes are fixed on a glass plate. However, they may be fixed on a silicon substrate instead.

As shown in FIG. 3, when helium gas 10 is flowed to accumulate in a space between the grounding electrode 1 and the RF current applying electrode 34, and the RF power source 9 was activated to apply 13.56 MHz RF output of about 10 W to the helium gas, helium plasma 33 was generated there under the atmospheric temperature. It has been known that DNA fragments are negatively charged in the plasma. Helium was used in this experiment because helium is ready to generate plasma under the atmospheric pressure. The output from the RF power source 9 may have a frequency of 2.45 GHz or a frequency in the range of microwave.

As is indicated by the experimental result shown in FIG. 4, voltage Vd necessary for maintaining helium plasma 33 depends on the gap distance Lg between the grounding electrode 1 and the RF current applying electrode 34. Voltage Vd was plotted while 3.56 MHz output of about 10 W was provided by the RF power source, with the RF current applying electrode 34 retaining DNA containing liquid paraffin 35 (open squares) or with the RF current applying electrode 34 being devoid of such DNA containing liquid paraffin (closed squares).

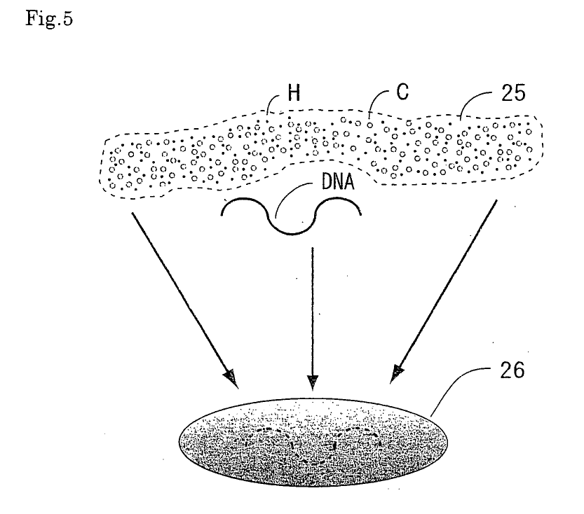

As is suggested by the principle enabling the introduction of DNA into a carbon cluster shown in FIG. 5, liquid paraffin vaporizes from the RF current applying electrode 34 to be broken up into carbon atoms (C) and hydrogen atoms (H) in plasma. DNA fragments are driven out via electric field developed on the surface of electrode 34, and drawn as negatively charged ions towards the plasma where they disperse to be attracted by the grounding electrode 1. On the other hand, carbon atoms (C) migrating towards the grounding electrode 1 entrap DNA fragments suspended along their course to form carbon clusters 26 around the entrapped DNA fragments. Thus, DNA doped carbon clusters 26 are formed on the surface of grounding electrode 1 in a soot-like deposit 24.

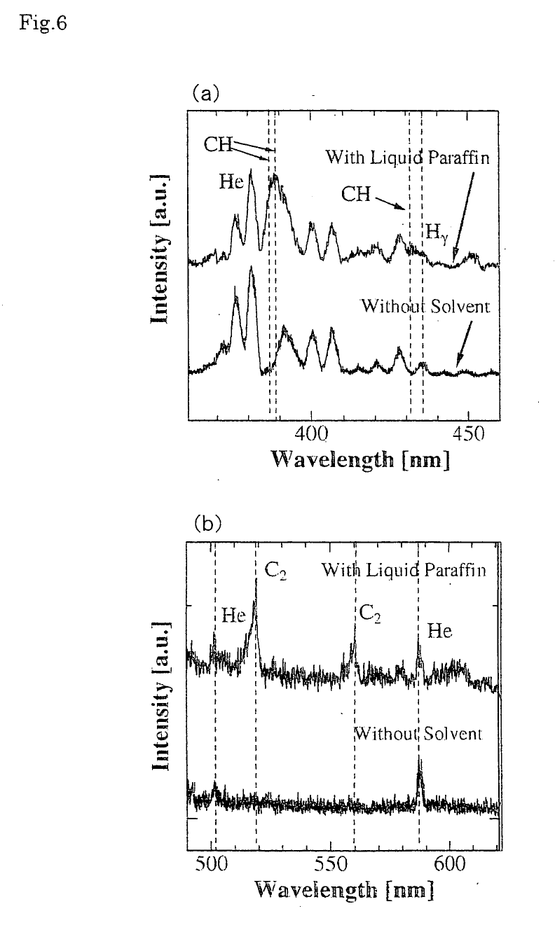

The results of spectroscopic analysis performed on plasma obtained from helium gas exposed to electric discharge are shown in FIG. 6. As shown in FIG. 6(a), CH peaks possibly ascribed to elements generated as a result of the decomposition of liquid paraffin occur at 387, 389 and 431 nm in terms of the wavelength of test beam. Similarly, as shown in FIG. 6(b), peaks ascribed to carbon molecule C₂ occur at 517 and 559 nm.

The soot-like deposit 24 was removed, transferred into organic solvent such as ethanol, washed by sonication, and subjected to electron microscopy. As a result, the existence of DNA doped carbon nanotubes was confirmed.

The above explanation has been given, for the sake of simplicity, with reference to an embodiment where hollow carbon nanotubes 2 were not arranged on the surface of grounding electrode 1. However, hollow carbon nanotubes 2 may be arranged on the surface of grounding electrode 1 as in Example 1. Then, DNA will be introduced into hollow carbon nanotubes 2 as well as is observed in Example 1.

1. An apparatus for producing a DNA doped carbon cluster comprising a radio frequency current applying electrode which is composed of a porous material or a wire mesh capable of retaining a solution containing DNA, a grounding electrode placed opposite to the radio frequency current applying electrode, and a power source for supplying a radio frequency output to the radio frequency current applying electrode, wherein the grounding electrode bears hollow carbon clusters on its surface.

2. An apparatus as described in claim 1 for producing a DNA doped carbon cluster wherein the radio frequency current applying electrode composed of a wire mesh comprises a laminated electrode composed of two kinds of wire meshes having different mesh densities.

3. An apparatus as described in claim 1 for producing a DNA doped carbon cluster wherein the solvent of the DNA containing solution is any one chosen from the group consisting of pure water, distilled water and liquid paraffin.

4. An apparatus for producing a DNA doped carbon cluster comprising a radio frequency current applying electrode composed of a porous material or a wire mesh capable of retaining a DNA containing solution, a grounding electrode placed opposite to the radio frequency current applying electrode, and a power source for supplying a radio frequency output to the radio frequency current applying electrode, wherein the solvent of the DNA containing solution is liquid paraffin.

5. An apparatus as described in claim 1 for producing a DNA doped carbon cluster, wherein the grounding electrode and the radio frequency current applying electrode are arranged on a silicon substrate.

6. An apparatus as described in claim 1 for producing a DNA doped carbon cluster, wherein the grounding electrode and the radio frequency current applying electrode are arranged on a glass substrate.

7. A method for producing a DNA doped carbon cluster comprising the step of applying a radio frequency output to a space between the radio frequency applying electrode retaining DNA and the grounding electrode placed opposite to the radio frequency applying electrode which bears hollow carbon clusters on its surface, so as to generate plasma in that space, thereby producing DNA doped carbon clusters.

8. A method for producing a DNA doped carbon cluster comprising the step of applying a radio frequency output to a space between the radio frequency applying electrode retaining DNA-containing liquid paraffin and the grounding electrode placed opposite to the radio frequency applying electrode so as to generate plasma in that space, thereby producing DNA doped carbon clusters.

9. A method as described in claim 7 for producing a DNA doped carbon cluster, wherein generation of plasma is achieved under the atmospheric pressure.

10. A DNA doped carbon cluster produced with an apparatus as described in claim 1.

11. A DNA doped carbon cluster produced by a method as described in claim 7.
